(19) United States
(12) Patent Application Publication
(10) Pub. No.: US 2012/0244612 A1
(43) Pub. Date: Sep. 27, 2012

(54) COMPOSTER

(75) Inventors: Kenton Good, Lancaster, PA (US); Kurtis Good, East Earle, PA (US); Daniel Gonzalez, East Earle, PA (US)

(73) Assignee: ROTARY COMPOSTERS, LLC, Denver, PA (US)

(21) Appl. No.: 13/418,000

(22) Filed: Mar. 12, 2012

Related U.S. Application Data
(60) Provisional application No. 61/454,671, filed on Mar. 21, 2011.

(51) Int. Cl. C12M 1/00

(52) U.S. Cl. 435/290.1

(57) ABSTRACT

In a rotary composter having a horizontally elongated rotatable cylindrical drum, material is fed from a hopper into the drum through an opening in a stationary end plate having an annular marginal area which overlaps, in sealing relationship, an annular intake end wall extending inward from the internal wall of the drum from a location adjacent to an intake end of the drum. At an exit end of the drum material passes through a central opening in an annular exit end wall and falls by gravity onto a cylindrical screen which is fixed to, and rotates with, the exit end wall. The annular end walls are in axial register with external trunnions on the drum which ride on pairs of supporting rollers. The exit end rollers allow for axial expansion of the drum. The interior wall the drum is lined with a rubber.

### CROSS-REFERENCE TO RELATED APPLICATION

This application claims priority on the basis from Provisional Application Ser. No. 61/454,671, filed Mar. 21, 2011.

### FIELD OF THE INVENTION

This invention relates to composting, and more particularly to a large-scale composting apparatus suitable for producing usable compost from plant and animal matter collected for example on a farm, at a meat packing plant, or at other facility where such materials accumulate.

### BACKGROUND OF THE INVENTION

In raising livestock and in meat packing, it is frequently necessary to dispose of the bodies of animals that die prematurely due to various causes. U.S. Pat. No. 6,703,234, granted on Mar. 9, 2004, for example describes a method and apparatus for composting animal bodies in which odors and pathogens are controlled by recirculation of air and gaseous products of the composting process.

Compost produced from animal carcasses, animal waste, vegetation, garbage, and other materials can be utilized as fertilizer. It is known to use a rotatable digesting drum in which material is introduced, mixed by rotation, and converted into usable fertilizer. In some cases, the composting operation is carried out as a batch process. In others, material is processed in the rotating drum continuously.

One of the problems encountered in continuous composting using a rotating drum is that of introducing material into the drum. Suitable automatic conveyors are expensive, and it is difficult to ensure reliable operation. On the other hand, manual introduction of material is both labor-intensive and unpleasant, and introduction of materials into a rotatable composter using machinery such as a front end loader is difficult especially because of the problem of avoiding spillage.

Another problem is that some materials, such as animal bones and some vegetable matter, will take longer to digest, and must be separated from the composted material in order to deliver a usable product.

Some composting applications can cause corrosion and premature deterioration of conventional composting equipment, necessitating frequent maintenance, and also early replacement of the rotating drum, which is the main component of the composter. Another problem is that composting material tends to slip on the side wall of a rotating drum, resulting in failure of composting media to turn over as desired. Another problem is that some compositing materials adhere to the wall of the rotating drum, resulting in non-uniform transit of material through the drum in a continuous composting operation.

### SUMMARY OF THE INVENTION

A composter according to the invention comprises an elongated cylindrical metal drum, typically twelve or more meters in length and three or more meters in diameter. The cylindrical wall of the drum is symmetrical about a horizontal axis, and the drum is slowly rotated about the axis by an indexing mechanism comprising a reciprocating pawl which cooperates with a set of ratchet teeth on the exterior of the drum, rotating the drum through several degrees of angle periodically. Material to be composted is fed into a hopper at an intake end of the drum, and converted within the drum into usable compost, which is delivered through an exit at the opposite end. A blower at the inlet end transfers air into the drum. Material moves axially through the drum as the drum rotates. Axial movement of the material takes place because the material tends to level as the drum rotates. Thus, as material is introduced at the inlet end, the level of the material in the drum rises, and a corresponding amount of material moves through the exit end.

One of the characteristic features of the composter according to the invention is the construction of its intake end. The material feed hopper is affixed to a stationary, preferably circular, plate having its center on the axis of rotation of the drum and located inside the drum a short distance from the intake end of the drum. A ring-shaped plate attached to the inner cylindrical surface of the drum cooperates with the circular plate to seal the intake end of the drum, thereby preventing composting material from falling out of the drum at the intake end. The material feed hopper is arranged to deliver material through an opening in the circular plate, the opening being located approximately centrally in the circular plate.

More particularly, in accordance with this first aspect of the invention, the composter comprises an elongated hollow drum having a substantially cylindrical wall symmetrical about a substantially horizontal axis and having an interior space. The drum is mounted for rotation on the substantially horizontal axis, and has axially spaced intake and exit ends. An annular intake end wall is fixed to the cylindrical wall and extends inward toward the drum axis from a location on the cylindrical wall adjacent to the inlet end of the drum. The intake end wall is rotatable with the drum and has a central opening. An annular exit end wall is fixed to the cylindrical wall and extends inward toward the drum axis from a location on the cylindrical wall adjacent to the exit end of the drum. This exit end wall is also rotatable with the drum. The end wall has a central exit opening for delivery of material from the interior space of the drum.

The composter also has a stationary end plate with an annular marginal area all of which overlaps a part of the intake end wall. The stationary end plate has a material feed opening in register with the central opening of the intake end wall. A feed hopper is fixed to the stationary end plate on the side thereof faced by the outer surface of the end plate, and includes a chute arranged to deliver material collected in the hopper through the material feed opening into the interior space of the drum. A drive mechanism is provided for rotating the drum about its axis.

A seal is provided between said annular marginal area of the stationary end plate and the part of the intake end wall overlapped by said marginal area. Preferably, the annular marginal area of the stationary end plate is located on the side of the annular intake end wall facing the interior space of the drum, and the seal is provided by a layer of grease in contact with both the annular marginal area of the stationary end plate and the part of the intake end wall overlapped by the marginal area of the end plate.

Another characteristic feature of the composter is a rotating sifting screen located at the exit end of the drum. The sifting screen is a generally cylindrical screen having openings of a suitable size to pass compost useful as fertilizer. The screen is affixed to an end plate that rotates with the drum. A cylindrical channel also affixed to the rotating end plate extends part-way into the cylindrical screen. The channel is coaxial with the drum and receives composted material through a central opening in the end plate. The diameter of the cylindrical screen is greater than that of the cylindrical channel, and consequently, material exiting the cylindrical channel drops by gravity onto the screen, and matter sufficiently fine to pass through the screen can be collected on a conveyor or in a suitable receptacle in which it can be hauled away from the composter for use. Coarse materials such as bones and undigested vegetable matter, which do not pass through the openings of the screen, move axially through the screen are delivered through an open end of the screen, from which they can be collected separately from the fine compost.

More particularly, a cylindrical screen is connected to and rotates with the exit end wall. This screen is coaxial with the central exit opening in the exit end wall, and has a diameter larger than the diameter of the central exit opening. The screen is being positioned to receive material delivered from the interior of the drum through the central exit opening, and has an end opening remote from the central exit opening, the end opening having a diameter substantially equal to the inner diameter of the cylindrical screen.

The drum is preferably mounted for rotation on two sets of supporting rollers, one set being adjacent to the intake end of the drum and the other set being adjacent to the exit end. External ring-shaped trunnions on the drum ride on the rollers. The axial length of the rollers at the exit end is greater than the width of the trunnion that engages them in order to allow for the axial expansion of the drum due to heat generated in the exothermic composting process.

More particularly, in accordance with this aspect of the invention, the cylindrical wall of the drum has a first external trunnion adjacent to its intake end and a second external trunnion adjacent to its exit end, and the composter includes first and second pairs of idler rollers rotatably supporting the drum. The first pair of rollers is in rolling engagement with the first external trunnion, and the second pair of rollers is in rolling engagement with the second external trunnion. The first external trunnion is restrained against axial movement, and the second trunnion is axially movable relative to the second pair of rollers, thereby allowing for axial expansion and contraction of the drum.

Still another characteristic feature of the composter is the positioning of the annular intake end wall or the annular exit end wall above a pairs of rollers on which the drum is supported. More particularly, in accordance with this aspect of the invention, at least one of said annular end walls meets the cylindrical wall of the drum at a location in axial register one of the external trunnions. Preferably, the annular intake end wall meets the cylindrical wall of the drum at a location in axial register with the first external trunnion, and the annular exit end wall meets the cylindrical wall of the drum at a location in axial register with the second external trunnion. In these positions, the annular end walls reinforce the drum, and thereby make it possible to reduce the overall weight of the drum.

Still another characteristic feature of the composter is a rubber lining covering the cylindrical inner wall of the drum. More particularly, the cylindrical wall of the drum has an inner surface coating composed of a rubber. The rubber lining, which is preferably sprayed-on, resists corrosion of the drum and also reduces adhesion of composting matter to the wall of the drum, thereby improving the uniformity of the residence time of the material in the drum.

Further features, objects, and advantages of the invention will be apparent from the following description when read in conjunction with the drawings.

### BRIEF DESCRIPTION OF THE DRAWINGS

FIG. 1 is an elevational view of the intake end of the composter, showing the feed hopper and the indexing mechanism;

FIG. 2 is a shortened side elevational view of the composter; and

FIG. 3 is an elevational view of the exit end of the composter.

### DETAILED DESCRIPTION OF THE PREFERRED EMBODIMENTS

As shown in the figures, the composter according to the invention includes as its main component a horizontally elongated cylindrical drum 10 mounted for rotation on a set of four rollers 12, 14, 16 and 18, on a substantially horizontal axis. A hopper 20 is provided at the intake end of the drum for the introduction of material to be composted into the drum. At the exit end, a short tubular extension 22 is provided for delivery of compost onto a cylindrical wire screen 24 which surrounds the tube.

The length of the drum is typically twelve or more meters and its diameter is typically 1.5 meters or more.

The composter is rotated at a slow rate, typically 2-3 revolutions per hour in steps of 3-10 degrees. The residence time for material in the composter, which is dependent primarily on the length of the drum and on the rate of introduction of material, is typically about 2-5 weeks.

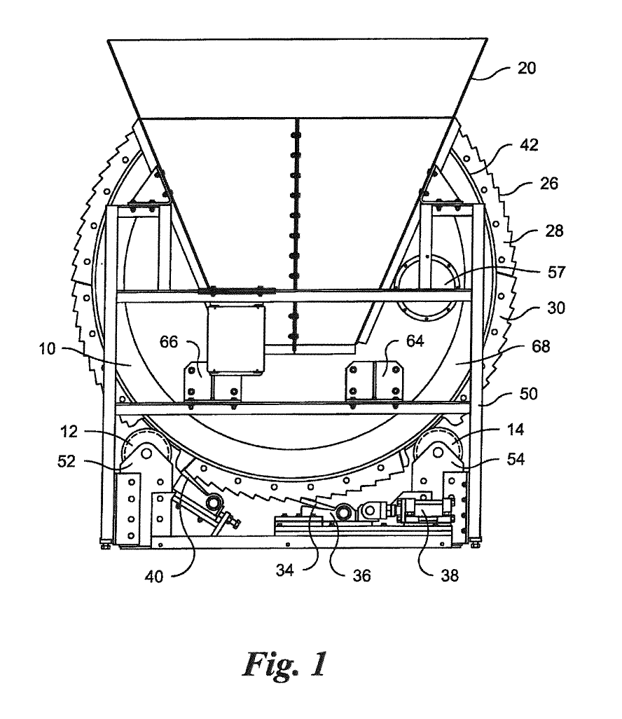

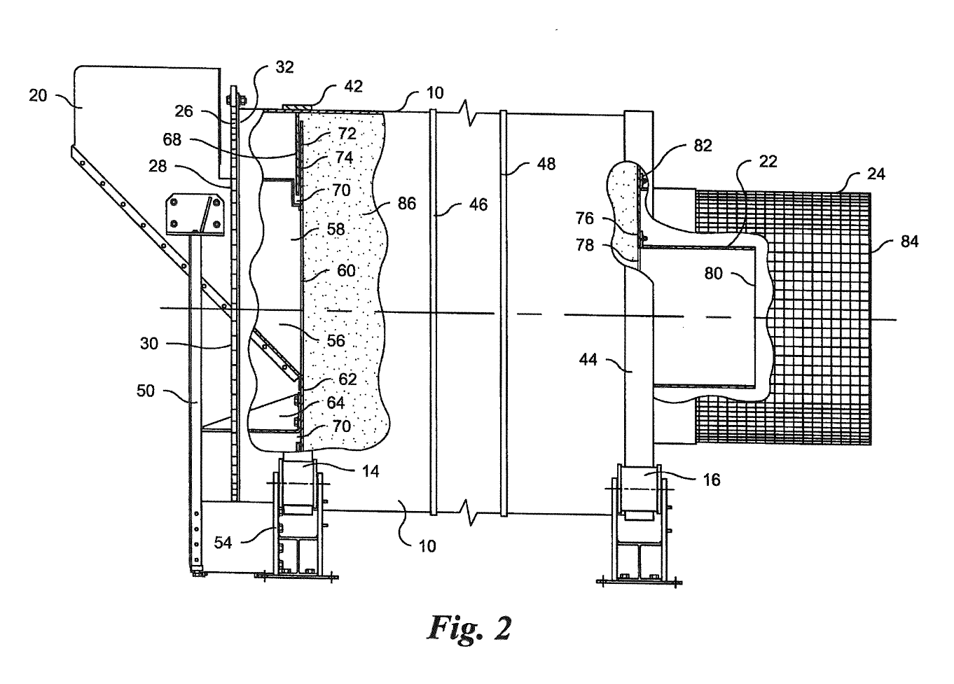

As shown in FIG. 1, the exterior of the drum is provided with a circular set of ratchet teeth 26 formed on arc-shaped segments, including segments 28 and 30, which are bolted to a flange 32 (FIG. 2) formed on or adjacent to the entry end of the drum.

The teeth are engaged by a pivoted pawl 34 on a sliding carriage 36. The pawl is urged against the ratchet teeth by a spring (not shown), and the carriage 36 is reciprocated in a direction tangent to the circle of ratchet teeth by the piston of a hydraulic cylinder 38 operated by hydraulic fluid supplied by a timer-operated pump-valve combination (not shown). A stationary spring-loaded pawl 40 is provided to prevent reverse rotation of the drum due to the unbalanced condition of the material inside the drum as the material accumulates on the left side of the interior of the drum (in FIG. 1) due to clockwise rotation.

As shown in FIG. 2, trunnions 42 and 44 formed on the drums, adjacent to the intake and exit ends respectively, engage the supporting rollers, trunnion 42 being engaged with rollers 12 and 14, and trunnion 44 being engaged with rollers 16 and 18. Flanges on rollers 12 and 14 are spaced by a distance substantially equal to the axial width of trunnion 42 and keep the intake end portion of the drum from moving axially. Flanges on rollers 16 and 18, however, are spaced from each other by a distance greater than the axial width of trunnion 44, as shown in FIG. 2, to allow for axial expansion and contraction of the drum resulting from temperature changes, which can range from ambient temperatures to considerably higher temperatures resulting from heat generated within the drum in the composting process. The temperature can range from below 0° C., depending on the climate of the region in which the composter is used, to above 70° C. The drum-supporting rollers 16 and 18 should be positioned so that their flanges are spaced from trunnion 44 in both axial directions at a suitable intermediate temperature, e.g., 35° C.

The drum can be reinforced not only by the roller-engaging trunnions 42 and 44, but by additional reinforcing rings, such as rings 46 and 48, provided on the exterior of the drum.

As shown in FIGS. 1 and 2, the feed hopper 20 is supported on a stationary frame 50, which is fixed to roller supports 52 and 54, on which rollers 12 and 14 are mounted respectively. The feed hopper has a chute 56, having a delivery opening 58 inside the drum. The chute 56 has an end opening in register with an opening 60 in a stationary end plate 62, which is also supported on stationary frame 50 by brackets 64 and 66 as well as by welds connecting the end plate to flanges on the end of chute 56. A blower 57 is provided on end plate for introducing air into the interior of the drum.

An annular intake end wall 68 is fixed, by welding, to the inside of the cylindrical wall of the drum so that it rotates with the drum. The intake end wall extends inwardly from the inner wall of the drum and has a central opening 70. The end wall 68 is overlapped by an annular marginal area 72 of the stationary end plate 62, which is preferably located adjacent to the side of the wall 68 that faces the exit end of the drum. A layer 74 of grease between the annular marginal area of the stationary end plate 62 and the overlapped part of the end wall 68 provides a seal that prevents the escape of composting matter through the intake end of the drum. The overlapping parts of the rotating intake end wall and the stationary end plate, and the layer of grease form a continuous annular seal surrounding the opening 60, through which matter to be composted enters the interior of the drum from the hopper. Preferably the bottom of the opening 60 is located a short distance below a level midway between the top and bottom of the cylindrical inner wall of drum 10. The annular intake end wall 68 is preferably axially in register with trunnion 42, i.e., it meets the inner wall of the drum at a location surrounded by trunnion 42. Because the wall 68 is in register with the trunnion 42, it reinforces the trunnion, making the trunnion better able to sustain the forces applied to it by the supporting rollers 12 and 14.

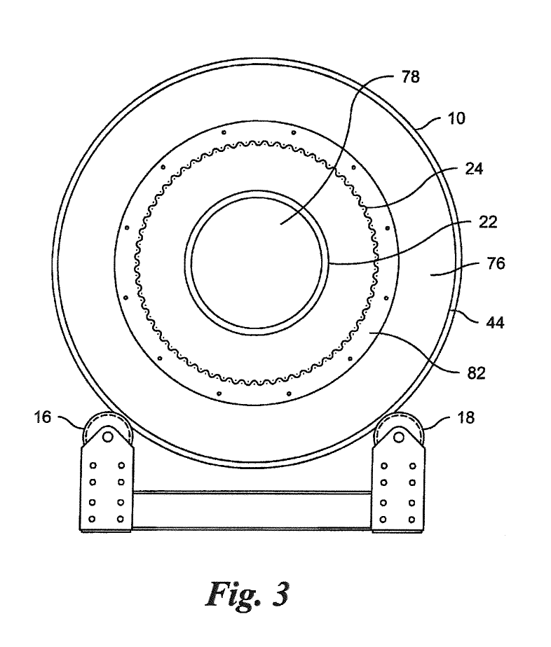

At the exit end of the drum, an annular exit end wall 76 (FIG. 3), having a central opening 78, is similarly in axial register with trunnion 44, and provides reinforcement to the trunnion.

As shown in FIGS. 2 and 3, the tubular extension 22 extends from wall 76 and rotates with wall 76. The extension is coaxial with the central opening 78 of wall 76 and the inner diameter of extension 22 is approximately equal to the diameter of opening 78 and the bottom of opening 78 is preferably approximately at the same level as the bottom of the hopper delivery opening 58. A delivery end 80 of the extension 22 is located within the cylindrical wire screen 24, which is affixed to wall 76 by a flange 82.

The wire screen is coaxial with extension 22 and has a diameter approximately twice that of the extension. As the drum rotates, matter introduced through the hopper at the intake end travels through the drum slowly while being converted into compost. Composted matter delivered through extension 22 falls onto the interior of the screen 24 and matter fine enough to pass through the screen can be collected in a receptacle or by a conveyor located below the screen. Coarse undigested matter will continue to move axially within the screen and can be collected separately by another receptacle or conveyor below the exit end 84 of the screen.

As shown in FIG. 2, the part of the cylindrical interior wall of the drum that is exposed to contact with composting matter is coated with a sprayed-on rubber lining 86. The sprayed-on rubber lining resists corrosion of the drum and also increases friction on the inner surface of the drum, which prevents slippage of the compost media, thereby ensuring that the material is tumbled as the drum rotates. By imparting a uniform frictional property to the inner surface of the drum, the rubber lining also prevents adhesion of some composting matter to the wall of the drum, thereby improving the uniformity of the residence time of the material in the drum. Finally, the rubber coating also insulates the drum, reducing loss of heat so that composting can take place more efficiently.

Advantages of the composter according to various aspects of the invention include the following. The composter can allow an operator to introduce new material at any desired time, and at any desired rate within a wide range, all while the composter is in continuous operation. Material delivered through the exit end of the composter can be dropped onto the interior of a cylindrical screen which rotates with the drum. The screen, which has an open end, automatically separates large undigested material from usable compost, delivering the compost through screen openings and delivering the larger materials through the open end. By positioning the annular intake and exit end walls in axial register with the external trunnions on the drum, the end walls can serve as reinforcements, making it possible to reduce the weight of the drum. The rubber lining reduces corrosion and improves uniformity of residence time. In addition, the relationship between the supporting rollers and the trunnions can allow for thermal expansion and contraction of the composter drum while ensuring that the sealing relationship between the intake end wall and the stationary end plate is maintained.

Various modifications can be made to the composter described. For example, the ratchet teeth by which the drum is driven can be formed on a continuous ring instead of on segments, and the ring, can be secured to a flange on the drum or fastened directly to the end of the drum by suitable means such as welding. Alternatively, individual ratchet teeth can be secured, by welding or other suitable means, to a flange or to an end of the drum

While an indexing drive is preferred, a continuously operating electric motor with a speed-reducing gear train can be used. Various different kinds of conveying devices can be used both to feed material to the intake end of the composter and to remove compost and waste from the exit end. While the axis of the drum is preferably horizontal, minor departures from horizontal rotation can be made without materially affecting the operation of the composter. Still other modifications can be made without departing from the scope of the invention as defined by the following claims.

What is claimed is:

1. A composter comprising:
an elongated hollow drum having a substantially cylindrical wall symmetrical about a substantially horizontal axis and having an interior space, said drum being mounted for rotation on said axis, and having axially spaced intake and exit ends;
an annular intake end wall fixed to the cylindrical wall and extending inward toward said axis from a location on the cylindrical wall adjacent to the inlet end of the drum, said intake end wall being rotatable with the drum and having a side facing the interior space of the drum and a central opening;
an annular exit end wall fixed to the cylindrical wall and extending inward toward said axis from a location on the cylindrical wall adjacent to the exit end of the drum and being rotatable with the drum, said exit end wall having a central exit opening for delivery of material from the interior space of the drum;
a stationary end plate having an annular marginal area all of which overlaps a part of the intake end wall, said stationary end plate having an inner surface facing the exit end of the drum, an outer surface facing away from the exit end of the drum, and an material feed opening in register with the central opening of the intake end wall;
a feed hopper fixed to the stationary end plate on the side thereof faced by said outer surface, and including a chute arranged to deliver material collected in the hopper through said material feed opening into the interior space of the drum; and
a drive mechanism for rotating said drum about said axis.

2. A composter according to claim 1, in which the annular marginal area of the stationary end plate is located on the side of the annular intake end wall facing the interior space of the drum.

3. A composter according to claim 1, including a seal between said annular marginal area of the stationary end plate and the part of the intake end wall overlapped by said marginal area.

4. A composter according to claim 3, in which said seal is a layer of grease in contact with both said annular marginal area and said part of the intake end wall overlapped by said marginal area.

5. A composter according to claim 1, in which the cylindrical wall of the drum has a first external trunnion adjacent to its intake end and a second external trunnion adjacent to its exit end, and including first and second pairs of idler rollers rotatably supporting said drum, the first pair of rollers being in rolling engagement with the first external trunnion, and the second pair of rollers being in rolling engagement with the second external trunnion.

6. A composter according to claim 5, in which the annular intake end wall meets the cylindrical wall of the drum at a location in axial register with the first external trunnion.

7. A composter according to claim 5, in which the annular exit end wall meets the cylindrical wall of the drum at a location in axial register with the second external trunnion.

8. A composter according to claim 5, in which the annular intake end wall meets the cylindrical wall of the drum at a location in axial register with the first external trunnion, and the annular exit end wall meets the cylindrical wall of the drum at a location in axial register with the second external trunnion.

9. A composter according to claim 5, in which the first external trunnion is restrained against axial movement, and in which the second trunnion is axially movable relative to the second pair of rollers, thereby allowing for axial expansion and contraction of the drum.

10. A composter according to claim 1, including a cylindrical screen connected to and rotatable with the exit end wall, said screen being coaxial with the central exit opening in the exit end wall, having a diameter larger than the diameter of the central exit opening, and being positioned to receive material delivered from the interior of the drum through said central exit opening, said cylindrical screen having an end opening remote from the central exit opening said end opening having a diameter substantially equal to the inner diameter of the cylindrical screen.

11. A composter according to claim 10, including a hollow cylindrical extension coaxial with said central exit opening and extending into the cylindrical screen from the exit end wall, said extension having an delivery opening in axial register with a part of the screen and having a diameter smaller than that of the screen, whereby material delivered from the interior of the drum through the extension falls by gravity onto the screen.

12. A composter according to claim 1 in which the cylindrical wall of the drum has an inner surface coating composed of a rubber.

13. A composter comprising:
an elongated hollow drum having a substantially cylindrical wall symmetrical about a substantially horizontal axis and having an interior space, said drum being mounted for rotation on said axis, and having axially spaced intake and exit ends;
a drive mechanism for rotating said drum about said axis;
a feed hopper adjacent the intake end of the drum, the hopper including a chute arranged to deliver material collected in the hopper into the interior space of the drum;
an annular exit end wall fixed to the cylindrical wall and extending inward toward said axis from a location on the cylindrical wall adjacent to the exit end of the drum and being rotatable with the drum, said exit end wall having a central exit opening for delivery of material from the interior space of the drum;
a cylindrical screen connected to and rotatable with the exit end wall, said screen being coaxial with the central exit opening in the exit end wall, having a diameter larger than the diameter of the central exit opening, and being positioned to receive material delivered from the interior of the drum through said central exit opening, said cylindrical screen having an end opening remote from the central exit opening said end opening having a diameter substantially equal to the inner diameter of the cylindrical screen.

14. A composter according to claim 13, including a hollow cylindrical extension coaxial with said central exit opening and extending into the cylindrical screen from the exit end wall, said extension having an delivery opening in axial register with a part of the screen and having a diameter smaller than that of the screen, whereby material delivered from the interior of the drum through the extension falls by gravity onto the screen.

15. A composter according to claim 13 in which the cylindrical wall of the drum has an inner surface coating composed of a rubber.

16. A composter comprising:
an elongated hollow drum having a substantially cylindrical wall symmetrical about a substantially horizontal axis and having an interior space, said drum being mounted for rotation on said axis, and having axially spaced intake and exit ends;
an annular intake end wall fixed to the cylindrical wall and extending inward toward said axis from a location on the cylindrical wall adjacent to the inlet end of the drum, said intake end wall being rotatable with the drum and having a side facing the interior space of the drum and a central opening;
an annular exit end wall fixed to the cylindrical wall and extending inward toward said axis from a location on the cylindrical wall adjacent to the exit end of the drum and being rotatable with the drum, said exit end wall having a central exit opening for delivery of material from the interior space of the drum;
a stationary end plate having an annular marginal area all of which overlaps a part of the intake end wall, said stationary end plate having an inner surface facing the exit end of the drum, an outer surface facing away from the exit end of the drum, and an material feed opening in register with the central opening of the intake end wall;
a feed hopper fixed to the stationary end plate on the side thereof faced by said outer surface, and including a chute arranged to deliver material collected in the hopper through said material feed opening into the interior space of the drum;
a drive mechanism for rotating said drum about said axis;
a first external trunnion adjacent to the intake end of the drum and a second external trunnion adjacent to the exit end of the drum; and
first and second pairs of idler rollers rotatably supporting said drum, the first pair of rollers being in rolling engagement with the first external trunnion, and the second pair of rollers being in rolling engagement with the second external trunnion;
in which at least one of said annular end walls meets the cylindrical wall of the drum at a location in axial register one of said external trunnions.

17. A composter according to claim 16, in which the annular intake end wall meets the cylindrical wall of the drum at a location in axial register with the first external trunnion.

18. A composter according to claim 16, in which the annular exit end wall meets the cylindrical wall of the drum at a location in axial register with the second external trunnion.

19. A composter according to claim 16, in which the annular intake end wall meets the cylindrical wall of the drum at a location in axial register with the first external trunnion, and the annular exit end wall meets the cylindrical wall of the drum at a location in axial register with the second external trunnion.

20. A composter according to claim 16 in which the cylindrical wall of the drum has an inner surface coating composed of a rubber.
